(19) United States
(12) Patent Application Publication
(10) Pub. No.: US 2003/0124130 A1
(43) Pub. Date: Jul. 3, 2003

(54) Proteomic analysis of tumors for development of consultative report of therapeutic options

(76) Inventor: Robert E. Brown, Winfield, PA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/325,793

(22) Filed: Dec. 19, 2002

Related U.S. Application Data
(60) Provisional application No. 60/345,309, filed on Jan. 2, 2002.

(51) Int. Cl. G01N 33/574; G06F 19/00; G01N 33/48; G01N 33/50; A61K 39/395

(52) U.S. Cl. 424/155.1; 435/7.23; 702/19

(57) ABSTRACT

The present invention provides methods of identifying potential therapeutic options for treatment of a tumor, and a consultative report providing the same.

### CROSS REFERENCE TO RELATED APPLICATIONS

This application claims the benefit of U.S. Provisional Application No. 60/345,309, filed Jan. 2, 2002, which is hereby incorporated by reference in its entirety.

### FIELD OF THE INVENTION

The present invention is directed to identifying potential therapeutic options for treatment of a tumor, and a consultative report providing the same.

### BACKGROUND OF THE INVENTION

Traditional diagnosis and tumor therapy begins with a patient presenting symptoms to a physician or through routine screening procedures on a patient presenting no symptoms. Biopsies may be taken and results obtained therefrom. A determination of cancer may be made in some cases and general therapeutic options including surgery and chemotherapy are selected. The diagnosis is routinely directed to the broad category of cancer and the therapy is generally not designed for the particular tumor involved. Rather, the therapy that is selected is often broad-based, i.e., based upon the cancer.

Recently, however, tumors have been analyzed in a more detailed and precise manner and differences among patients have been identified. For example, amplification of the human epidermal growth factor receptor (HER)-2/neu oncogene occurs in approximately 15 to 30% of invasive breast carcinomas. Slamon et al., Science, 1987, 235,177-182; Yu et al., Oncogene, 2000, 19, 6115-6121; and Hoang et al., Am. J. Clin. Pathol., 2000, 113, 852-859. This results in the overexpression of the corresponding protein receptor, leading to proliferative responses in the tumor cells via a tyrosine-kinase-mediated signal. Schechter et al., Nature, 1984, 312, 513-516; Akiyama et al., Science, 1986, 232, 1644-1646; and Wu et al., J. Clin. Lab. Anal., 1995, 9, 141-150. Clinically such overexpression and/or amplification portends a worse prognosis for the patients in terms of both a shorter disease-free interval and overall survival. Ross et al., Stem Cells, 1998, 16, 413-428. Thus, tailored treatments for some types of breast cancer can now be provided based upon a more detailed diagnosis.

Proteomics can be used to provide a more detailed diagnosis of tumors. Proteomics is the detection, quantification, cellular compartmentalization, and assessment of the functional state (phosphorylation) or functional grouping of proteins to create a molecular profile of tumor cells. Such analysis has identified molecular concomitants in the HER-2/neu protein-receptor-overexpressing and HER-2/neu gene-amplified human breast carcinoma cell line, SKBR-3. Brown, R. E., Annals of Clinical Labatory Science, 2002, 32, 12-21. Similar molecular characterization by proteomic analysis with therapeutic implications has been carried out for the Reed-Stemberg cell in Hodgkin's disease. Brown, R. E., et al., Annals of Clinical Laboratory Science, 2002, 32, 339-351. The specific proteins fall into one of the following categories: those involved in the generation of an extracellular signal and its transduction to the nucleus, thereby triggering molecular events at the genomic level including cellular proliferation; and those that are potential growth inhibitors or proapoptotic. A third category includes those proteins which can create an environment that promotes tumorigenesis.

Methodology for identifying potential therapeutic option(s) for treatment of a tumor and customized to the individual patient is needed. In particular, methods for identifying potential therapeutic option(s) for treatment of a tumor that are based upon an analysis of the molecular events occurring in such tumors, thereby providing additional insight into the pathogenesis of their growth, and that analyze a molecular profile of the tumor with defined pathways that present opportunities for therapeutic intervention, are needed.

### SUMMARY OF THE INVENTION

The present invention provides methods of identifying a potential therapeutic option for treatment of a tumor and customized to the individual patient. A cluster of protein expression for a proteomic profile of the tumor is correlated to at least one molecular pathway of the tumor. A potential therapeutic option is identified and is based upon the identified molecular pathway. A proteomic profile of the tumor can be performed prior to correlating the cluster of protein expression. The proteomic profile of the tumor can comprise an immunohistochemical or immunofluorescent analysis of the tumor.

The present invention also provides methods of generating a consultative report that identifies a potential therapeutic option for treatment of a tumor from a given patient. At least one molecular pathway identified as being a part of the pathology for the tumor from the patient is listed in the consultative report. At least one pharmaceutical agent that is directed to a specific molecular target involved in the identified molecular pathway is also listed in the consultative report. At least one molecular target involved in the molecular pathway of the tumor can also be listed in the consultative report. A score for at least one molecular pathway potentially involved in the pathology for the tumor can also be listed in the consultative report. The consultative report can also list the cellular compartment in which the molecular target was detected, illustrate its expression and provide a narrative interpretation, the name of the patient, the name of at least one physician, and an interpretation of the score for the molecular pathway, molecular target, or both.

### DESCRIPTION OF PREFERRED EMBODIMENTS

The present invention provides methods of identifying at least one potential therapeutic option for treatment of a tumor. A cluster of protein expression for a proteomic profile of the tumor is correlated to at least one molecular pathway of the tumor. A potential therapeutic option is identified and is based upon the identified molecular pathway. A proteomic profile of the tumor can be performed prior to correlating the cluster of protein expression. The proteomic profile of the tumor can comprise an immunohistochemical or immunofluorescent analysis of the tumor.

In some embodiments of the invention, a method of identifying at least one potential therapeutic option for treatment of a tumor is provided. The methods can also provide a plurality of potential therapeutic options for treatment of a tumor. As would be apparent to one of ordinary skill in the art, once armed with the teachings of the present application, the therapeutic options are potential, i.e., they are not guaranteed to be “cures” for treating the tumor. The therapeutic options will have been shown to provide or will be expected by one of ordinary skill in the art to provide at least some measurable palliative effect on the tumor based on in vitro data, in vivo data, animal model data, or clinical studies. The potential therapeutic options can be traditional chemotherapeutic options that have been and/or are now currently used to treat tumors, such as, for example, methotrexate, vincristine, prednisone, cytoxan, and adriemycin. Traditional chemotherapeutic options are described, for example, in Cancer Principles & Practice of Oncology (DeVita, et al. eds. 3^{rd} ed. J. B. Lippincott Co., Philadelphia, 1989). The potential therapeutic options can also be immunotherapeutic or homeopathic options that have been and/or are now currently used to treat tumors, such as, for example, trastuzumab sold under the trademark Herceptin™, anti-CD20 antibody known as rituximab and sold under the trademark Rituxan™, and BCG-immunization therapy. In addition, the potential therapeutic options can be newly developed chemotherapeutic, homeopathic or immunotherapeutic options, biological response modifiers or known agonists or antagonists of molecular pathways, such as, for example, STI571 sold under the trademark Gleevec™, and ZD1839 sold under the trademark IRESSA™.

Any tumor can be a target for treatment. Types of tumors include, but are not limited to, oligodendroglioma, ependymoma, meningioma, lymphoma, Ewing's sarcoma, chondrosarcoma, osteosarcoma, rhabdomyosarcoma, Schwannoma, medulloblastoma, breast, adrenal, pancreatic, parathyroid, pituitary, thyroid, anal, colorectal, esophageal, gall bladder, gastric, hepatoma, small intestine, cervical, endometrial, uterine, fallopian tube, ovarian, vaginal, vulvar, laryngeal, oropharyngeal, acute lymphocytic leukemia, acute myelogenous leukemia, chronic lymphocytic leukemia, chronic myogenous leukemia, hairy cell leukemia, mesothelioma, non small-cell lung carcinoma, small cell-lung carcinoma, AIDS-related lymphoma, cutaneous T-cell lymphoma, Hodgkin's disease, non-Hodgkin's, lymphoma, myeloma, penile, prostrate, melanoma, Kaposi's sarcoma, testicular, bladder, kidney, and urethral tumors. Samples of tumors, including tumor cells or tumor tissue, can be derived from, for example, biopsies taken from a patient.

In some embodiments of the invention, a cluster of protein expression for a proteomic profile of the tumor is correlated to at least one molecular pathway of the tumor. A “proteomic profile” of a tumor refers, for example, to the results obtained from the detection, quantification, determination of the cellular compartmentalization, assessment of the functional state (phosphorylation), functional grouping, and/or other characterization of proteins expressed in cells associated with the tumor. A proteomic profile of the tumor is generated prior to correlating a cluster of protein expression. In some embodiments of the invention, the proteomic profile of the tumor is generated at another location and/or time than is the correlation of a cluster of protein expression. For example, the proteomic profile can be generated at a laboratory or clinical site on one day (e.g., an out-patient clinic or lab) that is distinct from the site at which the cluster of protein expression is correlated another day (e.g., hospital, oncologist's office or laboratory). In other embodiments of the invention, the proteomic profile of the tumor is generated contemporaneously or near contemporaneously, in time and/or location, compared to correlating a cluster of protein expression. The patient can be any mammal, preferably human, that has been diagnosed as having a tumor or suspected of having a tumor. Alternately, the patient can have a tumor but yet be undiagnosed or not even suspected of having the tumor.

A proteomic profile of the patient's tumor can comprise an immunohistochemical or immunofluorescent analysis of the tumor. Immunohistochemical or immunofluorescent analysis of a tumor can be performed by any means known to the skilled artisan including, but not limited to, immunohistochemical or immunofluorescent staining, enzyme-linked immunosorbent assay (ELISA), in situ hybridization, Western blotting, confocal laser microscopy or scanning instrument, and the like, or any combination thereof. Indeed, any procedure for detecting the presence of a protein or other biological target can be used in the immunohistochemical or immunofluorescent analysis. The immunohistochemical or immunofluorescent analysis can further comprise determining the cellular location of a biological target in a cell of the tumor. Thus, immunohistochemical or immunofluorescent analysis can be performed on whole tumor cells, tumor cell extracts including the cytosol and/or organelles, membrane preparations from tumor cells, homogenized tumor cells, partially intact tumor cells, and the like.

The immunohistochemical or immunofluorescent analysis preferably comprises using a panel of at least about five, preferably at least about ten, or preferably at least about fifteen antibodies directed to different biological targets in the tumor. In some embodiments of the invention, antibodies to the following biological targets are used: Her-2/neu, Ki-67, p53, Cyclin D1, c-Jun, ER, PR, gp130, IL-6, IL-11, EGFR, TGF-α, farnesyl transferase, p21^{ras}, the latency associated peptide of TGF-β₁, TGF-βRII, bcl-2, and COX-2. Other biological targets that have been shown to play a role or are expected or suspected to play a role in tumor pathology include, but are not limited to, 14-3-3, Ab1, ACE, Adenomatous Polyposis Coli (APC colon cancer), Afx, Akt, AP-1, Apaf-1, Apaf2, Apaf3, APC (Anaphase Promoting Complex), APO-1, APO-2, APO-3, Apoptosis Signal-Regulating Kinase, ASK1, AT1, ATF, ATM, ATR, Bad, Bak, Bax, Bcl-1, Bcl-w, Bcl-x1, BID, BRCA1, BRCA2, Bub1, CA2+/Calmodulin Kinase II/IV, CAD, Cadherin, Calsequestrin, CAP4, CASPASE1, CASPASE10, CASPASE11, CASPASE2, CASPASE3, CASPASE4, CASPASE6, CASPASE7, CASPASE8, CASPASE9, Cathepsin D, Caveolin, CBP, CD31(Pecam-1), CD40, CD40L, CD95, CD95L(ligand for CD95), cdc25a, cdc25b, cdc25c, Cdc34, Cdc42, Cdk2, Cdk4, Cdk6, Cdk7, Cdk8, c-Fos, chk1, Chk2/hcds1, c-myc, Cox-2, Csk, Cullin-1, Cyclin A, Cyclin B1, Cyclin, Cyclin D2, Cyclin D3, Cyclin E, Cyclin E2, cytochrome C, DAXX, DFF40, DFF45, DNA-PK, Dp-1, E2f, EGF, Eif2a, Elk-1, ErbB3, ErbB4, ERK1, ERK2, ERK3, ERK5, Estrogen Receptor, FADD, FAF1, FAK, FAP-1, Fas, FasL, Fkhrl1, FLICE, FLICE2, Fodrin, Frizzled, Fyn, gadd45, GAP, Glucocorticoid Receptor, Grb2, Growth Hormone Receptor, Gsk3b, HDAC, Her3, Her4, Histone Deacetylase 4, Histone eacetylase 5, Histone H3, HMG-1, HMG-2, Hsp90, Histone H4, ICAD, ICE, IKKa, Ikkb, IKKg, IL-2, IL-b 2R, Irs-1, Jak1, Jak2, Jkk1, JNK Oun n-terminal kinase), Jnk1, KSR-1 (kinase suppressor of Ras), Ku, lamin b1, Lamin a, lamin b2, Lck, MACH, MADD, Mannose 6-phosphate Receptor, MAP2, MAPK, MAPKK, MAPKKK, Mch2, Mch3, Mch4, Mch5, Mch6, mcl-1, MCM2, MCM3, MCM5, MDM-2, Mek, MEK3, MEK4, MEK6, MEK7, MIHA/XIAP, MIHB/cIAP1, MIHC/cIAP2, MLH1, MNDA, Mnk1, Mnk2, mre11, MSH2, MSk1, Mucin2, MyoD1, myt1, Nck, NFAT3, NF-Kb, nibrin, Nik, NLK, N-myc (neuronal), P/CAF, P107, P14ARF, P15, P16INK4, P19ARF, P21/waf1/cip1, P27, P300, P38 MAP Kinase, P45, P48-IFN a signaling, P51, P63, P73, Pak1, Paxillin, PCNA (Proliferating cell nuclear antigen), PDGF, PDGFR, Pdk-1, PI3K, PKB, PKC, PKR, PLCg, Poly(ADP)-Ribose Polymerase PARP), Pp130, PP2A, par, PTEN, Pyk2, Rab9, Rac, RAD50, Rad51, Rad9, Raf1, RAIDD, Ral, Ras, Rb, Replication protein A, Rho a, RIP, Rsk-1, Rsk-2, Rsk-3, SAPK (stress activated inase), She, Skp2, Smac/Diablo, SMAD1, SMAD2, SMAD3, Smad4, SnoN, SODD, Sos-1, Src, STAT1, STAT3, Stat5, STAT6, Survivin, Syk, TAB1, TAK1, Talin, Tcf4, TGFb, TNFR, TNFR1, TNFR2, TNFB, TNFa, TRADD, TRAF1, TRAF2, Traf-3, TRAF3, TRAF4, TRAF5, TRAF6, Trail,TyK2, VDAC, weel, Wilm's Tumor Protein-1 (WT-1), wnt, b-Catenin, and b-TRCP. Antibodies to these biological targets, and in some instances to their phosphorylated state, are readily available from private or commercial sources such as, for example, Oncogene Research Products (San Diego, Calif.), Calbiochem (San Diego, Calif.), Santa Cruz Biotechnology, Inc. (Santa Cruz, Calif.), and Cell Signaling Technology, Inc. (Beverly Mass.).

Protein expression can be measured via the immunohistochemical or immunofluorescent analysis applied to individual tissue sections or to tissue microarrays, or using other methods that are readily known to those of ordinary skill in the art, and a cluster of protein expression identified. In some embodiments of the invention, a cluster of protein expression can be a single protein. More often, however, a cluster of protein expression includes a plurality of different proteins. For example, immunohistochemical or immunofluorescent analysis may reveal over-expression, or expression of a mutated form, of several proteins. Alternately, immunohistochemical or immunofluorescent analysis may reveal under-expression or no expression of a wild-type protein or proteins or of a wild type protein or proteins in their activated (phosphorylated) state. In any event, the over-expression, under-expression, no expression, or expression of a mutated form, or a combination thereof, of one or more proteins are referred to as a cluster of protein expression. The cluster of protein expression is correlated to at least one molecular pathway of the tumor. The terms “molecular pathway” and “physiological pathway” are understood by those of skill in the art to be interchangeable. Such correlation can be performed by humans or computers and can be based on, for example, existing databases or sources of information regarding molecular pathways of tumor cells (so-called data-mining). Such databases or sources of information regarding molecular pathways of tumor cells can be continually updated to include newly discovered molecular pathways and biological targets or proteins. The terms “molecular targets” and “biological targets” refer to any biological entity that is affected in some way by a therapeutic or pharmaceutical agent that is part of a potential therapeutic option to provide at least some measurable palliative effect on a tumor based on in vitro data, in vivo data, animal model data, or clinical studies. The literature, as well as internet databases and web sites, are replete with biological targets involved in tumors as well as the molecular pathways in which they are involved. Indeed, the National Cancer Institute's web site is replete with information regarding the pathology of cancers and tumors. The molecular pathways can be distinct or can be inter-related with other molecular pathways. The molecular pathways can also be named based upon the starting signal, the end signal, the enzymes involved therein, or by any other rationale. A number of molecular pathways known to the skilled artisan have been identified in tumor cells.

A panel of antibodies directed to about fifteen or more biological targets, for example, may reveal over-expression of, for example, three of the biological targets and expression of, for example, one mutant biological target. The over-expressed biological targets and expression of one mutant biological target forms a cluster of protein expression. Further, this particular cluster of protein expression may be found, for example, in multiple molecular pathways of tumors cells as determined theoretically or physically in experimental studies and may be reported in the literature or via the internet. Thus, the proteins forming the cluster of protein expression are correlated with the multiple molecular pathways.

A number of molecular pathways are described below and involve numerous biological targets. In some embodiments of the invention, the following molecular pathways can be correlated to a cluster of protein expression in a tumor. The cytokines IL-6 and IL-11 and their corresponding receptors complex with a shared signal-transducing subunit, gp 130, to activate the JAK-STAT pathway. Briscoe et al., Current Biology, 1994, 4, 1033-1035. The binding of activated STATs to DNA gamma activated sequence (GAS) elements leads to the induction of transcription and molecular expression of certain genes. In addition, IL-6 through gp130 signaling can activate other pathways that involve downstream effector molecules, including Ras. Briscoe et al., Current Biology, 1994, 4, 1033-1035. Moreover, there is evidence that IL-6 can induce tyrosine phosphorylation of c-erbB-2 (HER-2/neu) and that the latter can form a complex with the gp130 subunit of the IL-6 receptor in an IL-6 dependent manner, leading in turn to signaling through the mitogen-activated protein (MAP) kinase pathway. Qiu et al., Nature, 1998, 393, 83-85. Similarly, the coexpression of EGFR (c-erbB-1) and its ligand, TGF-α, in the SKBR-3 cell line could initiate protracted signaling in an autocrine fashion, and tumoral proliferation through the MAP or extracellular signal-regulated kinase (ERK) pathway. Umekita et al., Int. J. Cancer, 2000, 89, 484-487; Nakagami et al., Hypertension, 2001, 37, 581-586; Martinez-Lacaci et al., Int. J. Cancer, 2000, 88, 44-52; Sizemore et al., Gastroenterology, 1999, 117, 567-576; and Reddy et al., Int. J. Cancer, 1999, 82, 268-273. An important intermediary step in this process is the activation of p21^{ras} via farnesylation. Sizemore et al., Gastroenterology, 1999, 117, 567-576. Further, there appears to be collaboration between c-erbB-2 (HER-2/neu) and c-erbB-1 (EGFR) in promoting farnesylation, given the previous observation by Asslan and co-workers (Biochem. Biophys. Res. Commun., 1999, 260, 699-706) that epidermal growth factor stimulates 3-hydroxy-3-methylglutaryl-coenzyme A reductase expression via the erbB-2 pathway in SKBR-3 cells (this enzyme represents an early but key step leading to the biosynthesis of farnesyl pyrophosphate, the substrate for farnesyl transferase). Sweetman et al., In: The Metabolic Basis of Inherited Disease, 6th ed (Scriver C R, Beaudet A L, Sly W S, Valle D, Eds), McGraw-Hill, New York, 1989, pp 804-805; and Cohen et al., Biochem. Pharmacol., 1995, 49, 839-845. The prior demonstration of heterodimers of c-erB-1 and c-erbB-2 in the SKBR-3 cell line accords with such collaboration. Goldman et al., Biochemistry, 1990, 29, 11024-11028.

Proteomic analysis by immunohistochemistry has identified molecular concomitants in HER-2/neu positive breast carcinoma that outline possible collaborations with the EGFR and JAK/STAT system and signal transduction through farnesylation of p21^{ras} in promoting the tyrosine-kinase-mediated proliferations of such tumors. Similar profiling of each individual patient's tumor could identify such pathogenic pathways and thereby point to specific opportunities for customized therapeutic intervention. Reinforcement for such an approach can be found in the genomic studies of Perou and associates, who painted molecular portraits of human breast tumors using complementary DNA microarrays. Notably, they found that the tumors show great variation in their patterns of gene expression and that many different sets of genes show mainly independent patterns of variation. Perou et al., Nature, 2000, 406, 747-752. Further, these investigators utilized immunohistochemical detection of proteins encoded by a particular gene in a cluster to identify the cell type involved. Perou et al., Nature, 2000, 406, 747-752; and Perou et al., Proc. Natl. Acad. Sci. USA, 1999, 96, 9212-9217.

These data coincide with the observations in the literature in suggesting signal transduction through farnesylated p21ras as part of the pathogenesis of tyrosine-kinase-mediated proliferation in HER-2/neu protein-receptor-positive breast carcinoma. Involvement with the EGFR and JAK/STAT systems in these molecular events are also likely. Future therapeutic considerations should include downregulators of c-erb-B1 (EGFR) and -B2 (HER-2) receptor expressions, inhibitors of tyrosine kinase and farnesylation, and activators of growth innibitory/proapoptotic pathways.

In some embodiments of the inventive methods, at least one potential therapeutic option is identified and is based upon the molecular pathway identified from the cluster of protein expression. In some embodiments of the invention, only one potential therapeutic option is identified. In other embodiments of the invention, a plurality (i.e., more than one, e.g., two or more) of potential therapeutic options is identified. Identifying a potential therapeutic option can comprise identifying a pharmaceutical agent that is targeted to a protein involved in the molecular pathway identified. In some embodiments of the invention, the pharmaceutical agent is targeted to one of the proteins that forms the cluster of protein expression. In other embodiments of the invention, the pharmaceutical agent is targeted to one of the proteins involved in the molecular pathway identified but is not directed specifically to one of the proteins that forms the cluster of protein expression. For example, proteins X and Y may be identified from the proteomic profile to form a cluster of protein expression. Proteins X and Y may, in turn, be correlated to molecular pathway B, in which proteins G, F and H are also involved. The pharmaceutical agent can be directed to proteins X and/or Y, but can also be directed to proteins G, F and/or H. Thus, the pharmaceutical agent is targeted to any protein or biological target involved in the molecular pathways previously identified and correlated to the cluster of protein expression. The pharmaceutical agent need not be specific for a particular target. The pharmaceutical agent can be any compound that has been shown to be useful or is expected to be useful in the treatment of a tumor. Pharmaceutical agents are well known to the skilled artisan. Sources of pharmaceutical agents include, for example, the Physician's Desk Reference, scientific and clinical literature, internet web sites, The Food and Drug Administration publications, and the like.

The present invention also provides methods of generating a consultative report that identifies a potential therapeutic option for treatment of a tumor from a patient. The consultative report can be a hand-written report or can be electronic and can, for example, be output by computer. At least one molecular pathway identified as being a pathology for the tumor from the patient can be listed in the consultative report. At least one pharmaceutical agent that is directed to a molecular target involved in the identified molecular pathway can also be listed in the consultative report. At least one molecular target involved in the molecular pathway for the tumor can also be listed in the consultative report. A score for at least one molecular pathway potentially involved in the pathology for the tumor can also be listed in the consultative report. The consultative report can also list or graphically depict the cellular compartment in which the biological target was detected, a narrative interpretation, the name of the patient, the name of at least one physician, and an interpretation of the score for the molecular pathway, molecular target, or both.

In one embodiment of the inventive methods, at least one molecular pathway identified as being a part of the pathology for the tumor from the patient is listed. In addition, in some embodiments of the invention, at least one pharmaceutical agent that is directed to a biological target involved in the physiological pathway identified is listed. In some embodiments of the invention, a plurality of molecular pathways potentially involved in the pathology for the tumor is listed or graphically depicted. The molecular pathways and pharmaceutical agents are described further above.

In some embodiments of the invention, the methods of generating a consultative report further comprise listing at least one biological target involved in the molecular pathway for the tumor. In some embodiments of the invention, biological targets include, but are not limited to, Her-2/neu, Ki-67, p53, Cyclin D1, c-Jun, ER, PR, gp130, IL-6, IL-11, EGFR, TGF-α, farnesyl transferase, p21^{ras}, TGF-β₁, TGF-βRII, bcl-2, and COX-2. Other biological targets are described above. In other embodiments of the invention, a plurality of biological targets potentially involved in the molecular pathway for the tumor are listed. In some embodiments, the cellular compartment in which the biological target was detected is listed.

In some embodiments of the invention, the methods of generating a consultative report further comprise listing a score for at least one of the plurality of molecular pathways potentially involved in the pathology for the tumor. The score is a value corresponding to the level of involvement of a physiological pathway in the pathology for the tumor. The score is preferably a measure of a proteomic profile of the tumor. Any type of scoring system can be used. For example, a low score is given to molecular pathways for which no cluster of protein expression has been correlated. A high score is given to a physiological pathway for which a cluster of protein expression has been correlated.

In some embodiments of the invention, the methods of generating a consultative report further comprise listing a score for at least one of the plurality of biological targets potentially involved in the molecular pathway for the tumor. The score is a value corresponding to the level of involvement of at least one of the plurality of biological targets potentially involved in the molecular pathway for the tumor. The score is preferably a measure of a proteomic profile of the tumor. Any type of scoring system can be used. For example, a low score is given to biological targets for which little or no expression or over-expression has been shown. A high score is given to biological targets for which expression or over-expression has been shown.

In some embodiments of the invention, the methods of generating a consultative report further comprise listing an interpretation of the score for the molecular pathway, biological target, or both. The interpretation can describe the scoring system itself, the actual scores for a particular patient, the scores for control tests, as well as optimal or desirable scores, or any combination thereof.

In some embodiments of the invention, the methods of generating a consultative report further comprise listing a narrative interpretation. The narrative interpretation can comprise at least one of the following: literature citation or summary regarding the type of tumor, literature citation or summary regarding at least one molecular pathway identified as being a pathology for the tumor from the patient, literature citation or summary regarding at least one pharmaceutical agent that is directed to a biological target involved in the physiological pathway identified, literature citation or summary regarding side-effects of listed pharmaceutical agents, literature citation or summary regarding the biological target, and literature citation or summary regarding precedent for treatment with the pharmaceutical agent(s) listed, summary of expected prognosis, or any combination thereof. The narrative interpretation can be generated or aided, in part, by information downloaded from the internet from a number of web sites including, for example, the Food and Drug Administration and various pharmaceutical companies. The information contained within the narrative interpretation can, thus, be continually updated via searching and mining the internet and other sources.

In some embodiments of the invention, the methods of generating a consultative report further comprise listing the name of the patient and/or the name of at least one physician.

In order that the invention disclosed herein may be more efficiently understood, examples are provided below. It should be understood that these examples are for illustrative purposes only and are not to be construed as limiting the invention in any manner. Further, although the below example is directed to a breast tumor, similar methodology can be used for any other tumor. In addition, the disclosures of each patent, patent application, and publication cited or described in this document are incorporated herein by reference in their entirety.

### EXAMPLES

### Example 1

### HER-2 and Molecular Pathways in Breast Carcinoma

Slides containing sections of three pelleted human breast carcinoma cell lines (DAKO HercepTest™) and expressing HER-2/neu protein-receptor scored at 3+ (SKBR-3), 1+(MDA-175) and 0 (MDA-231), respectively, were reacted in immunohistochemical procedures for the detection of the following antigens: HER-2, estrogen receptor (ER), progesterone receptor (PR), Ki-67, cyclin D1, c-Jun, epidermal growth factor receptor (EGFR), transforming growth factor (TGF)-α, components of the JAK/STAT signal transduction pathway (gp130, interleukin (IL)-6, and IL-11), p21^{ras}, farnesyl transferase (FT), and potential growth inhibitory/proapoptotic and antiapoptotic-related proteins (latency-associated peptide (LAP) of TGF-β1, TGF-β receptor (R) II, p53, and bcl-2 and cyclooxygenase (COX)-2). Immunoreactivities were graded using bright-field microscopy on a scale of 0 to 3+.

Human Breast Cancer Cell Lines

Slides, each containing sections of three pelleted, formalin-fixed and paraffin-embedded human breast carcinoma cell lines—SKBR-3, MDA-175 and MDA-231, respectively—were obtained as part of the standardized DAKO HercepTest™ (DAKO Corporation, Carpinteria, Calif.).

Immunohistochemistry

A panel of antibodies was assembled to detect the following antigens in the aforementioned cell lines: HER-2/neu protein receptor, Ki-67, cyclin D1, c-Jun, ER, PR, EGFR, TGF-α, gp130, IL-6, IL-11, p21^{ras}, FT, LAP of TGF-β1, TGF-βRII, p53, bcl-2, and COX-2.

Anti-HER-2/neu polyclonal antibody (code #K5205; HercepTest™) was used to confirm the cellular distribution and the immunoreactivity for HER-2/neu protein receptor expression as stated for the individual cell lines.

Mouse monoclonal anti-human Ki-67 antibody (clone MIB-1; DAKO) was used to detect the corresponding antigen, a non-histone nuclear protein associated with all active phases in the cell cycle (G1, S, G2, and M).

Mouse monoclonal anti-human cyclin D1 antibody (clone DCS-6; DAKO) was used to detect the corresponding antigen, a protein that positively regulates the progression of the cell cycle in the G1 to S phase.

Mouse monoclonal anti-c-Jun antibody with human immunoreactivity (clone 3; BD Transduction Laboratories, Becton, Dickinson and Company, USA) was used to assess the nuclear expression of the c-Jun antigen, a protein product of its corresponding proliferation-associated, immediate-early gene and an essential component of the activator protein transcription factor (AP-1).

Mouse monoclonal anti-human ER antibody (clone 1D5; DAKO) was used to assess the expression of the corresponding antigen, a largely intranuclear protein that mediates the action of estrogenic hormones.

Mouse monoclonal anti-human PR antibody (clone PgR636; DAKO) was used to assess the expression of the corresponding antigen, an intranuclear protein that functions as a transcription factor, mediating the effect of progestogenic hormones.

Mouse monoclonal anti-human EGFR antibody (clone 2-18C9; DAKO EGFR pharmDx) was used to detect the plasmalemmal expression of the corresponding antigen, a transmembrane protein with an extracellular domain which after interactions with EGF or TGF-a generates a tyrosine-kinase-mediated signal resulting in cell proliferation. Reynolds et al., Nature, 1981, 292, 259-262.

Mouse monoclonal anti-human TGF-α antibody (clone 9426.21, IgG1; R&D Systems, Inc. Minneapolis, Minn.) was used to detect the corresponding antigen, a protein (cytokine) with high affinity for EGFR.

Mouse monoclonal anti-human gp130 antibody (clone 28118.11, IgG1; R&D Systems) was used to detect the corresponding antigen, a transmembrane protein that is the signal-transducing subunit for IL-6 and IL-11. Briscoe et al., Current Biology, 1994, 4, 1033-1035.

Mouse monoclonal anti-human IL-6 and IL-11 antibodies (clones 1936.14, IgG2b and 22315.1, IgG2a,k, respectively; R & D Systems) were used to assess the expression of the corresponding antigens, proteins (cytokines) whose signals are mediated through gp130 and the JAK/STAT signal transduction pathway. Briscoe et al., Current Biology, 1994, 4, 1033-1035.

Mouse monoclonal anti-human p21^{ras} antibody (clone NCC-RAS-001; DAKO) was used to detect the corresponding antigen, a protein encoded by the H-ras gene and which functions as a guanine nucleotide-binding (G) protein involved in signal transduction.

Rabbit polyclonal anti-human FTα antibody (catalog #sc-487; Santa Cruz Biotechnology, Inc., USA) was used to detect the corresponding antigen, a peptide mapping at the carboxy terminus of the 49 kD, α subunit common to farnesyl and geranylgeranyl transferases, that catalyze the prenylation and thereby, activation of ras-related proteins.

Goat polyclonal antibody reactive with LAP of human TGF-β1 (catalog #AB-246-NA; R&D Systems) was used. This antibody against LAP has been shown to react with latent TGF-β1 in immunohistochemical applications.

Rabbit polyclonal anti-human TGF-β RII antibody (catalog #sc-220; Santa Cruz Biotechnology) was used to assess the expression of the corresponding antigen, a glycoprotein designed to mediate a signal from active TGF-β.

Mouse monoclonal anti-human p53 antibody (clone BP53 12-1; BioGenex, San Ramon, Calif.) was used to assess the expression of the corresponding antigen, a primarily intranuclear protein that can exist in both wild-type and mutant forms, the latter reflecting a specific genetic change in malignant breast cancer.

Mouse monoclonal anti-human bcl-2 antibody (clone 124; DAKO) was used to investigate the expression of the corresponding antigen, a protein that plays a key role in the inhibition of apoptosis.

Rabbit polyclonal anti-human PGHS-2 (product #PG 27B; Oxford Biomedical Research, Inc., Oxford, Mich.) was used to assess the expression of the C-terminus of the COX-2 isoenzyme, a protein that is involved in the pathway leading to bcl-2 synthesis, thereby reducing apoptosis. Fosslien, Ann. Clin. Lab. Sci., 2000, 30, 3-21.

The general immunohistochemical procedure has been previously described. Brown, Ann. Clin. Lab. Sci., 1997, 27, 329-337. Positive controls using established immunoreactive tissues and a negative control utilizing the three cell lines provided in the HercepTest™ were shown to react appropriately.

Analysis of Immunostains

The scoring of the overexpression of HER-2/neu protein receptor was carried out in accordance with criteria outlined in the standardized procedure. All other immunoreactivities in the three cell lines were scored from 0 (negative) to 3+ positivity using bright-field microscopy.

Results

Immunoreactivities for HER-2/neu protein-receptor expression in the three cell lines were confirmed at 3+ for SKBR-3, 1+ for MDA-175 and 0 for MDA-231. Circumferential plasmalemmal positivity characterized the expression in SKBR-3 cells.

Proteomic analysis by immunohistochemistry and scoring by bright-field microscopy revealed the following commonalities among these three breast carcinoma cell lines: relatively high proliferation indices with 54, 40 and 61% of nuclei showing Ki-67 antigen expression; absent (0) chromogenic signals for ER and PR, and positive signals (1 to 3+) for IL-6, IL-11, EGFR, TGF-α, TGF-β1 (LAP), TGF-βR II, FT, p21ras and p53. Strong intranuclear immunopositivities for cyclin D1 and c-Jun antigens, respectively, were evident in the MDA-231 cell line but absent or rare (0 to ±) in SKBR-3. Conversely, gp130 antigen was scored at 1+ in the SKBR-3 cell line but only weakly expressed (±) in MDA-231 cells. Finally, bcl-2 and COX-2 were detected in the cytoplasm of the MDA-231 cells but absent from the SKBR-3 cell line. Individual scores for each of these protein analytes according to cell line and cellular compartment are detailed in Table 1.

TABLE 1
Assessment of potential molecular concomitants in HER-2/neu protein-
receptor-positive breast carcinoma using proteomic analysis by
immunohistochemistry.*

| | Breast Carcinoma Cell Line | | |
|---|---|---|---|
| Protein Analyte | SKLBR-3 | MDA-175 | MDA-231 |
| HER-2/neu† | 3+ | 1+ | 0 |
| Ki-67§ | 54% | 40% | 61% |
| p53§¶ | 3+ | 1+ | 3+ |
| Cyclin D1§ | 0 | 2+ | 2+ |
| c-Jun§¶ | ± | 1+ | 3+ |
| ER§ | 0 | 0 | 0 |
| PR§ | 0 | 0 | 0 |
| gp130†‡ | 1+ | 2+ | + |
| IL-6¶ | 2+ | 3+ | 3+ |
| IL-11¶ | 3+ | 3+ | 3+ |
| EGFR† | 2+ | 2+ | 3+ |
| TGF-α¶ | 2+ | 2+ | 1+ |
| Famesyl Transferase¶ | 3+ | 3+ | 3+ |
| p21^{ras}¶ | 1+ | 1+ | 1+ |
| TGF-β₁ (LAP)¶ | 3+ | 3+ | 2+ |
| TGF-βRII¶ | 3+ | 3+ | 3+ |
| bc1-2¶ | 0 | ± | 1+ |
| COX-2¶ | 0 | 1+ | 2+ |

*Grading of immunoreactivity on a scale of 0 to 3+ (± = faint reactivity). Cellular compartment of immunoreactivity:
†plasmalemmal;
‡vesicular;
§nuclear;
¶diffuse cytoplasmic.

The finding of a relatively high proliferation (Ki-67) index at 54% (Friedrich et al., Anticancer Research, 1999, 19, 3349-3353) in the SKBR-3 cell line is consistent with the known ability of the activated ras/ERK pathway to promote DNA synthesis and cellular proliferations. Proteomic analysis in this example has identified components of the JAK/STAT, EGFR, p21ras and farnesylation pathways that provide opportunities for collaboration with over-expressed HER-2/neu protein-receptor in triggering cellular proliferation in this subset of human breast carcinomas.

As a potential counter to the relatively high cell cycle activity (Ki-67 antigen expression) in all three of these human breast carcinoma cell lines, there is the co-expression of TGF-βRII and the latency-associated peptide of TGF-β1. In general and following conversion from its latent to active form, TGF-β1 can complex with TGF-βRII to inhibit epithelial cell proliferation. Fosslien et al., Ann. Clin. Lab. Sci., 2000, 30, 3-21; Miyazono et al., Ann. NY Acad. Sci., 1990, 593, 51-58; Zugmaier et al., Ann. NY Acad. Sci., 1990, 593, 272-275; Wakefield et al., Cancer Treatment & Research, 1992, 61, 97-136; and Ko et al., J. Cell. Physiol., 1998, 176, 424-434. The exposure of the human breast carcinoma cell lines, MDA-231 and SKBR-3 to TGF-β1 effected growth inhibition in several studies. Zugmaier et al., Ann. NY Acad. Sci., 1990, 593, 272-275; Wakefield et al., Cancer Treatment & Research, 1992, 61, 97-136. In a related experiment, tamoxifen incubation with MDA-231 cells induced TGF-β1 protein and was associated with a G1/G0 cell cycle blockade and with induction of apoptosis. Perry et al., Br. J. Cancer, 1995, 72, 1441-1446. Further, the demonstration by Arteaga and colleagues of growth stimulation in MDA-231 cells following exposure to anti-transforming growth factor beta antibody provided evidence that cultured human breast cancer cells can utilize endogenously produced TGF-β as an autocrine negative growth factor. Arteaga et al., Cell Growth & Differentiation, 1990, 1, 367-374.

In addition to their potential roles in influencing events at the genomic level to promote or inhibit growth, respectively, IL-11 and the latency-associated peptide of TGF-β1 could alter the extratumoral environment to promote tumorigenesis of human breast carcinoma. Specifically, IL-11 from MDA-231 cells has been implicated in the osteolytic bony metastasis of experimental animals by virtue of its ability to promote osteoclastogenesis. Morinaga et al., Int. J. Cancer, 1997, 71, 422-428; and Girasole et al., J. Clin. Invest., 1994, 93, 1516-1524. In so doing, IL-11 could facilitate the growth and expansion within osseous tissues of those human breast carcinomas that produce it. The latency-associated peptide of TGF-β1 might contribute to tumorigenesis by down-regulating host immune surveillance and, in particular, by interfering with natural killer cell and cytotoxic T-lymphocyte functions. Malygin et al., Scand. J. Immunol, 1993, 37, 71-76; Harthun et al., J. Immunother., 1998, 21, 85-94; Nagy et al., Cancer Immunol. Immunother., 1998, 45, 306-312; Wakefield et al., Cancer Treatment & Research, 1992, 61, 97-136. It appears that both of these lymphocytic cell types possess an intrinsic ability to convert latent TGFβ-1 to its active form, leading to the down-regulation of immune surveillance. Wakefield et al., Cancer Treatment & Research, 1992, 61, 97-136; and Wallick et al., J. Exp. Med., 1990, 172, 1777-1784.

By utilizing the molecular concomitants identified in this study and drawing on the observations of other investigators, one skilled in the art can envision a pathogenic sequence for the increased proliferative activity that is amenable to therapeutic intervention in HER-2/neu protein-receptor-positive breast carcinoma at multiple stages. In particular, because activation of p21^{ras} via farnesylation is key to the downstream transduction of growth factor receptor signaling, the use of farnesyl transferase or farnesyl diphosphate synthase inhibitors such as L739, 749 and aminobisphosphonates, respectively, are possible therapeutic options. Sizemore et al., Gastroenterology, 1999, 117, 567-576; Hortobagyi et al., Seminars in Oncology, 1999, 26, 11-20; Scharovsky et al., J. Biomed. Sci., 2000, 7, 292-298; Bulus et al., Neoplasia, 2000, 2, 357-364; Luckman et al., J. Bone & Mineral Research, 1998, 13, 581-589; and Bergstrom et al., Arch. Biochem. Biophys., 2000, 373, 231-241. In this regard, it is noteworthy that L739, 749 has been shown to cause tumor regressions of carcinomas in transgenic mice (Lavelle, Comptes Rendus des Seances de la Societe de Biologie et de Ses Filiales, 1997, 191, 211-219), and bisphosphonates have reduced the metastatic tumor burden of MDA-231 cells in bone in nude mice (Sasaki et al., Cancer Res., 1995, 55, 3551-3557; Sasaki et al., Int. J. Cancer, 1998, 77, 279-285) and have been shown to have direct anti-tumor effects, causing apoptosis in human breast cancer cell lines (Senaratne et al., Br. J. Cancer, 2000, 82, 1459-1468). Moreover, bisphosphonates are routinely used to treat the osteolytic lesions of metastatic breast carcinoma. Hillner, Oncology, 2000, 10, 250-253; Diel, Medizinische Klinik, 2000, 95, 9-18; and Oura, Breast Cancer, 2000, 7, 307-310. Alternatively, inhibitors of 3-hydroxy-3methylglutaryl-coenzyme A (HMG CoA) reductase such as lovastatin also may achieve the same effect by interfering in an earlier stage of farnesyl synthesis. Rubins et al., Am. J. Resp. & Crit. Care Med., 1998, 157, 1616-1622. Correspondingly, the treatment of MDA-231 cells with mevinolin, an inhibitor of HMG CoA reductase, resulted in decreased DNA-synthesis and depressed their proliferation. Wejde et al., Anticancer Research, 1992, 12, 317-324. Attempting to interrupt any autocrine signaling initiated by TGF-α and p75 activations (Sizemore et al., Gastroenterology, 1999, 117, 567-576; Lupu et al., Proc. Nat. Acad. Sci. USA, 1992, 89, 2287-2291) of EGFR (c-erbB-1) and HER-2/neu (c-erbB-2), respectively, also would seem to be a logical approach. This may be achieved by the use of agents such as ZD 1839, an EGFR-tyrosine kinase inhibitor (Ciardiello et al., Clin. Cancer Res., 2000, 6, 2053-2063; and Meric et al., Bulletin du Cancer, 2000, 87, 873-876) and trastuzumab, a monoclonal antibody directed against the extracellular domain of the HER-2/neu receptor (Ross et al., Stem Cells, 1998, 16, 413-428; Baselga et al., Semin. Oncol., 1999, 26, 78-83; Anon, Herceptin® (trastuzumab) anti-HER-2/neu monoclonal antibody (package insert) Genentech, South San Francisco, Calif., 1998; and Slamon et al., N. Engl. J. Med., 2001, 344, 783-792). Both therapies are currently being used in clinical settings. Meric et al., Bulletin du Cancer, 2000, 87, 873-876; Slamon et al., N. Engl. J. Med., 2001, 344, 783-792. Finally, to promote apoptosis of the tumor cells and to improve host immune surveillance, one might consider using retinoids such as 4-hydroxyphenylretinamide (4-HPR) to convert the latency-associated peptide of TGF-β1 to its active form. Herbert et al., Nutrition & Cancer, 1999, 34, 121-132; and Jinno et al., Carcinogenesis, 1999, 20, 229-236. Retinoids also have been shown to down-regulate the expression of HER-2/neu protein receptor (Grunt et al., Br. J. Cancer, 1998, 78, 79-87; Offterdinger et al., Biochem. Biophys. Res. Commun., 1998, 251, 907-913; and Schneider et al., Breast Cancer Research & Treatment, 1999, 58, 171-181), possibly to reduce the incidence of second breast malignancies in premenopausal women (Camerini et al., J. Clin. Oncol., 2001, 19, 1664-1670), and to enhance natural killer cell functional activity (Villa et al., Br. J. Cancer, 1993, 68, 845-850). Although seemingly paradoxic, the combined effect of c-erb B-1 (EGFR) and c-erb B-2 (HER-2/neu) would be to promote the action of retinoids through the up-regulation of retinoic acid receptor-alpha production. Flicker et al., Cancer Letters, 1997, 115, 63-72. The finding herein of absent bcl-2 protein and COX-2 antigen expression in the SKBR-3 cell line suggests that intrinsic opposition to this apoptotic approach may be minimal. Fosslien et al., Ann. Clin. Lab. Sci., 2000, 30, 3-21. The molecular pathways created by functional grouping of the proteins described above provide for a pathogenetic sequence leading to growth of the tumor that involves: 1) heterodimerization of HER-2/neu (c-erbB-2) and TGF-α-activated EGFR(c-erbB-1); 2) upregulation of HMG-CoA reductase; 3) farnesylation of p21^{ras}; and 4) downstream signaling through the extracellular signal-regulated kinase (ERK) culminating in nuclear DNA synthesis and cellular proliferation. Additional tumorigenic effects can include down-regulation (−) of TGF-βRII by farnesylated p21^{ras} and of natural killer (NK) cell activity by the latency-associated peptide (LAP) of TGF-β1. Opportunities for therapeutic intervention into this pathogenetic sequence include: a) trastuzumab (anti-HER-2/neu antibody); b) ZD 1839, an inhibitor of c-erbB-1 tyrosine kinase; c) lovastatin-like agents to inhibit HMG-CoA; d) aminobisphosphonates or farnesyl transferase inhibitors (e.g., L739, 749) to interrupt farnesylation; and e) retinoids (such as 4-hydroxyphenyl retinamide, 4-HPR) to down-regulate c-erbB expression and to activate (+) latent TGF-β1, leading to apoptosis and enhanced NK cell activity.

Various modifications of the invention, in addition to those described herein, will be apparent to those skilled in the art from the foregoing description. Such modifications are also intended to fall within the scope of the appended claims.

The disclosures of each patent, patent application, and publication cited or described in this document are hereby incorporated by reference in their entirety.

What is claimed is:

1. A method of identifying at least one potential therapeutic option for treatment of a tumor comprising the steps of:
correlating a cluster of protein expression for a proteomic profile of said tumor to at least one molecular pathway of said tumor; and
identifying at least one potential therapeutic option based upon said molecular pathway identified.

2. The method of claim 1 further comprising the step of generating a proteomic profile of said tumor prior to said correlating a cluster of protein expression.

3. The method of claim 1 wherein said proteomic profile of said tumor comprises an immunohistochemical or immunofluorescent analysis of said tumor.

4. The method of claim 3 wherein said immunohistochemical or immunofluorescent analysis comprises using a panel of at least five antibodies directed to different molecular targets in said tumor.

5. The method of claim 3 wherein said immunohistochemical or immunofluorescent analysis comprises using a panel of at least ten antibodies directed to different biological targets in said tumor.

6. The method of claim 3 wherein said immunohistochemical or immunofluorescent analysis comprises using a panel of at least fifteen antibodies directed to different biological targets in said tumor.

7. The method of claim 4 wherein said immunohistochemical or immunofluorescent analysis further comprises determining the cellular location of a biological target in a cell of said tumor.

8. The method of claim 4 wherein said molecular targets are selected from the group consisting of Her-2/neu, Ki-67, p53, Cyclin D1, c-Jun, ER, PR, gp130, IL-6, IL-11, EGFR, TGF-α, farnesyl transferase, p21^{ras}, the latency-associated peptide of TGF-β₁, TGF-βRII, bcl-2, and COX-2.

9. The method of claim 1 wherein said tumor is selected from the group consisting of oligodendroglioma, ependymoma, meningioma, lymphoma, Ewing's sarcoma, chondrosarcoma, osteosarcoma, rhabdomyosarcoma, Schwannoma, medulloblastoma, breast, adrenal, pancreatic, parathyroid, pituitary, thyroid, anal, colorectal, esophageal, gall bladder, gastric, hepatoma, small intestine, cervical, endometrial, uterine, fallopian tube, ovarian, vaginal, vulvar, laryngeal, oropharyngeal, acute lymphocytic leukemia, acute myelogenous leukemia, chronic lymphocytic leukemia, chronic myogenous leukemia, hairy cell leukemia, mesothelioma, non small-cell lung carcinoma, small cell-lung carcinoma, AIDS-related lymphoma, cutaneous T-cell lymphoma, Hodgkin's disease, non-Hodgkins, myeloma, penile, prostrate, melanoma, Kaposi's sarcoma, testicular, bladder, kidney, and urethral tumors.

10. The method of claim 1 wherein said identifying a potential therapeutic option comprises identifying a pharmaceutical agent that is targeted to a protein involved in said molecular pathway identified.

11. A method of identifying at least one potential therapeutic option for treatment of a tumor comprising the steps of:
correlating a cluster of protein expression for a proteomic profile comprising an immunohistochemical or immunofluorescent analysis of said tumor to at least one physiological pathway of said tumor, wherein said immunohistochemical or immunofluorescent analysis comprises using a panel of at least five antibodies directed to different biological targets selected from the group consisting of Her-2/neu, Ki-67, p53, Cyclin D1, c-Jun, ER, PR, gp130, IL-6, IL-11, EGFR, TGF-α, farnesyl transferase, p21^{ras}, the latency-associated peptide of TGF-β₁, TGF-βRII, bcl-2, and COX-2 in said tumor; and
identifying at least one potential therapeutic option based upon said physiological pathway identified.

12. A method of generating a consultative report that identifies at least one potential therapeutic option for treatment of a tumor from a patient comprising the steps of:
listing at least one molecular pathway identified as being a part of the pathology for said tumor from said patient; and
listing at least one pharmaceutical agent that is directed to a biological target involved in said molecular pathway identified.

13. The method of claim 12 wherein said tumor is selected from the group consisting of oligodendroglioma, ependymoma, meningioma, lymphoma, Ewing's sarcoma, chondrosarcoma, osteosarcoma, rhabdomyosarcoma, Schwannoma, medulloblastoma, breast, adrenal, pancreatic, parathyroid, pituitary, thyroid, anal, colorectal, esophageal, gall bladder, gastric, hepatoma, small intestine, cervical, endometrial, uterine, fallopian tube, ovarian, vaginal, vulvar, laryngeal, oropharyngeal, acute lymphocytic leukemia, acute myelogenous leukemia, chronic lymphocytic leukemia, chronic myogenous leukemia, hairy cell leukemia, mesothelioma, non small-cell lung carcinoma, small cell-lung carcinoma, AIDS-related lymphoma, cutaneous T-cell lymphoma, Hodgkin's disease, non-Hodgkin's lymphoma, myeloma, penile, prostrate, melanoma, Kaposi's sarcoma, testicular, bladder, kidney, and urethral tumors.

14. The method of claim 12 further comprising the step of listing a plurality of molecular pathways potentially involved in said pathology for said tumor.

15. The method of claim 12 further comprising the step of listing at least one biological target involved in said molecular pathway for said tumor.

16. The method of claim 15 wherein said at least one biological target is selected from the group consisting of Her-2/neu, Ki-67, p53, Cyclin D1, c-Jun, ER, PR, gp130, IL-6, IL-11, EGFR, TGF-α, farnesyl transferase, p21^{ras}, the latency-associated peptide of TGF-β₁, TGF-βRII, bcl-2, and COX-2.

17. The method of claim 12 further comprising the step of listing a plurality of biological targets potentially involved in said molecular pathway for said tumor.

18. The method of claim 14 further comprising the step of listing a score for at least one of said plurality of molecular pathways potentially involved in said pathology for said tumor.

19. The method of claim 17 further comprising the step of listing a score for at least one of said plurality of biological targets potentially involved in said molecular pathway for said tumor.

20. The method of claim 18 wherein said score is a value corresponding to the level of involvement of a molecular pathway in said pathology for said tumor.

21. The method of claim 19 wherein said score is a value corresponding to the level of involvement of said at least one of said plurality of biological targets in said molecular pathway for said tumor.

22. The method of claim 20 wherein- said score is a measure of a proteomic profile of said tumor.

23. The method of claim 21 wherein said score is a measure of a proteomic profile of said tumor.

24. The method of claim 12 further comprising the step of listing a narrative interpretation.

25. The method of claim 24 wherein said narrative interpretation comprises at least one of literature citation or summary regarding said tumor, literature citation or summary regarding said at least one molecular pathway identified as being a pathology for said tumor from said patient, literature citation or summary regarding said at least one pharmaceutical agent that is directed to a biological target involved in said molecular pathway identified, literature citation or summary regarding side-effects of listed pharmaceutical agents, literature citation or summary regarding said biological target, summary of expected prognosis, and literature citation or summary regarding precedent for treatment with said at least one pharmaceutical agent.

26. The method of claim 12 further comprising the step of listing the name of said patient.

27. The method of claim 12 further comprising the step of listing the name of at least one physician.

28. The method of claim 12 further comprising the step of listing an interpretation of said score for said molecular pathway, biological target, or both.

29. The method of claim 12 further comprising the step of listing the cellular compartment in which said biological target was detected.
